(19) United States
(12) Patent Application Publication
(10) Pub. No.: US 2023/0172746 A1
(43) Pub. Date: Jun. 8, 2023

(54) Body-Worn Drainage Bag Storage Device

(71) Applicant: Lynda Smith, Randleman, NC (US)

(72) Inventor: Lynda Smith, Randleman, NC (US)

(21) Appl. No.: 17/746,276

(22) Filed: May 17, 2022

Related U.S. Application Data
(60) Provisional application No. 63/285,569, filed on Dec. 3, 2021.

(51) Int. Cl. A61F 5/44; A41F 9/02

(52) U.S. Cl. CPC A61F 5/4408; A41F 9/025

(57) ABSTRACT

The present invention relates to a body-worn drainage bag storage device that is primarily comprised of a body that receives a drainage bag, wherein the body is further comprised of a front surface, a rear surface, and at least one loop that receives at least one band. A body-worn drainage bag can be placed within an interior space of the body, wherein the bag can then be secured within the body by at least one fastener on the front surface and rear surface of the body. The body is further comprised of at least one loop that receives at least one band. The band is preferably an elastic band that can be worn around the upper body and/or waist of the user.

### CROSS-REFERENCE TO RELATED APPLICATION

The present application claims priority to, and the benefit of, U.S. Provisional Application No. 63/285,569, which was filed on Dec. 3, 2021 and is incorporated herein by reference in its entirety.

### FIELD OF THE INVENTION

The present invention relates generally to the field of body-worn drainage bags. More specifically, the present invention relates to a body-worn drainage bag storage device that is primarily comprised of a body that receives a drainage bag, wherein the body is further comprised of a front surface, a rear surface, and at least one loop that receives at least one band. A body-worn drainage bag can be placed within an interior space of the body, wherein the bag can then be secured within the body by at least one fastener on the front surface and rear surface of the body. The body is further comprised of at least one loop that receives at least one band. The band is preferably an elastic band that can be worn around the upper body and/or waist of the user. In an additional embodiment, the device is comprised of a second band that can also be worn around the thigh of the user. In this manner, the at least one strap allows the device to be secured to the body of a user. In this manner, the device conceals the drainage bag such that a user can wear normal clothes that otherwise would not be able to conceal the drainage bag from view. Accordingly, the present disclosure makes specific reference thereto. Nonetheless, it is to be appreciated that aspects of the present invention are also equally applicable to other like applications, devices and methods of manufacture.

### BACKGROUND

A cholecystostomy procedure is used to drain fluid buildup in the gall bladder. During the procedure, a drainage catheter is placed in the gall bladder and is connected to a drainage bag outside of the body of the patient. The bag must then be periodically emptied as it becomes full. However, gall bladder drainage bags (and other drainage bags of the like used for similar procedures) can be extremely uncomfortable and also embarrassing to wear. The bag can further hang down from the waist of a user and drag on the floor. As a result, the bag may be stepped on, which can cause leaking of the bag or serious injury to the user. In addition, wearing normal clothes may be impossible with a gall bladder drainage bag due to the inability of the clothing to conceal the bag. As a result, a user may suffer from decreased self-esteem and self-consciousness as a result of the inability to wear normal clothing.

Therefore, there exists a long-felt need in the art for a device that allows a user to conceal a gall bladder drainage bag or other similar drainage bag of the like from view. There also exists a long-felt need in the art for a body-worn drainage bag storage device that can be worn on the body of a user and wherein the device conceals a gall bladder drainage bag or other similar drainage bag of the like from view. Further, there exists a long-felt need in the art for a body-worn drainage bag storage device that can be worn on the body of a user, that conceals a gall bladder drainage bag or other similar drainage bag of the like from view, and also allows user to wear normal clothes that still conceal the drainage bag.

The subject matter disclosed and claimed herein, in one embodiment thereof, comprises a body-worn drainage bag storage device. The device is primarily comprised of a body that receives a drainage bag, wherein the body is further comprised of a front surface, a rear surface, and at least one loop that receives at least one band. A body-worn drainage bag can be placed within an interior space of the body, wherein the bag can then be secured within the body by at least one fastener on the front surface and rear surface of the body. The body is further comprised of at least one loop that receives at least one band. The band is preferably an elastic band that can be worn around the upper body and/or waist of the user. In an additional embodiment, the device is comprised of a second band that can also be worn around the thigh of the user. In this manner, the at least one strap allows the device to be secured to the body of a user. In this manner, the device conceals the drainage bag such that a user can wear normal clothes that otherwise would not be able to conceal the drainage bag from view.

In this manner, the body-worn drainage bag storage device of the present invention accomplishes all of the forgoing objectives and provides a device that allows a user to conceal a gall bladder drainage bag or other similar drainage bag of the like from view. Further, the device can be worn on the body of a user wherein the device conceals a gall bladder drainage bag or other similar drainage bag of the like from view. As a result, the device allows a user to wear normal clothes that still conceal the drainage bag.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a body-worn drainage bag storage device. The device is primarily comprised of a body that receives a drainage bag, wherein the body is further comprised of a front surface, a rear surface, and at least one loop that receives at least one band. The body is preferably manufactured from a durable fabric that is preferably waterproof or water-resistant. The body may also be opaque, semi-transparent, or transparent and may be comprised of a plurality of indicia such as, but not limited to: patterns, logos, emblems, images, symbols, designs, letters, words, characters, animals, advertisements, brands, etc. The body can also be any color known in the art and is preferably rectangular in shape. An interior space of the body further receives a body-attached drainage bag such as, but not limited to: a gall bladder drainage bag.

The rear surface of the front surface and the front surface of the rear surface are each further comprised of at least one fastener. In the preferred embodiment, the fastener of the rear surface is a hook fastener and the fastener of the front surface is a loop fastener. However, in differing embodiments the fastener may be any fastener known in the art such as, but not limited to: magnetic, snap button, zipper, adhesive, etc. such that the front surface can be secured to the rear surface. As a result, a drainage bag can be secured within the interior space of the body while the device is being used by securing the fasteners of each surface to one another. The rear surface of the rear surface may also have a moisture-wicking, fabric, padded layer that does not irritate the skin of a user when the device is worn by a user.

The top edge of the body is further comprised of at least one loop. The loop receives at least one band that is preferably made from an elastic material, wherein the rear surface of the band is further comprised of the same padded material as the padded layer of the rear surface. In one embodiment, the band is one-size-fits-all and can stretch to fit a variety of users. However, in another embodiment, the band may be a two-piece band that has at least one fastener that allows a user to tighten or loosen the band around their waist and/or upper body. In another embodiment of the device, the bottom edge of the body is also comprised of at least one loop that receives at least one band of the same construction. In this manner, said band of the bottom edge functions as a leg-worn band that offers additional support to the body of the device.

To use the device, a user first places a drainage bag within the interior space of the body. Then, the user can secure both fasteners of the front surface and rear surface together to secure the bag within the body. A user can then secure the at least one band around their waist and/or upper body. Further, in an embodiment of the device with two bands the second band can also be secured around the thigh of the user. Once the bag is within the body of the device and the device is secured on the user, the user can then get dressed as normal. Because of the streamlined shape of the body, the bag is concealed from view and the user can also wear normal clothes due to this streamlined shape. In addition, the device prevents the bag from dragging or falling on the ground.

Accordingly, the body-worn drainage bag storage device of the present invention is particularly advantageous as it provides a device that allows a user to conceal a gall bladder drainage bag or other similar drainage bag of the like from view. Further, the device can be worn on the body of a user wherein the device conceals a gall bladder drainage bag or other similar drainage bag of the like from view. As a result, the device allows a user to wear normal clothes that still conceal the drainage bag. In this manner, the body-worn drainage bag storage device overcomes the limitations of existing body-worn drainage bags and methods of wearing said drainage bags known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a body-worn drainage bag storage device of the present invention in accordance with the disclosed architecture;

FIG. 2 illustrates a top view of one potential embodiment of a body-worn drainage bag storage device of the present invention in accordance with the disclosed architecture; and

FIG. 3 illustrates a perspective view of one potential embodiment of a body-worn drainage bag storage device of the present invention while being worn by a user in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that allows a user to conceal a gall bladder drainage bag or other similar drainage bag of the like from view. There also exists a long-felt need in the art for a body-worn drainage bag storage device that can be worn on the body of a user and wherein the device conceals a gall bladder drainage bag or other similar drainage bag of the like from view. Further, there exists a long-felt need in the art for a body-worn drainage bag storage device that can be worn on the body of a user, that conceals a gall bladder drainage bag or other similar drainage bag of the like from view, and also allows user to wear normal clothes that still conceal the drainage bag.

The present invention, in one exemplary embodiment, is comprised of a body-worn drainage bag storage device primarily comprised of a body that receives a drainage bag, wherein the body is further comprised of a front surface, a rear surface, and at least one loop that receives at least one band. The body is preferably manufactured from a durable fabric that is preferably waterproof or water-resistant, wherein the body may also be opaque, semi-transparent, or transparent and may be comprised of a plurality of indicia such as, but not limited to: patterns, logos, emblems, images, symbols, designs, letters, words, characters, animals, advertisements, brands, etc. The body can also be any color known in the art and is preferably rectangular in shape. An interior space of the body further receives a body-attached drainage bag such as, but not limited to: a gall bladder drainage bag.

The rear surface of the front surface and the front surface of the rear surface are each further comprised of at least one fastener, wherein the fastener of the rear surface is preferably a hook fastener and the fastener of the front surface is preferably a loop fastener. However, in differing embodiments the fastener may be any fastener known in the art such as, but not limited to: magnetic, snap button, zipper, adhesive, etc. such that the front surface can be secured to the rear surface. As a result, a drainage bag can be secured within the interior space of the body while the device is being used by securing the fasteners of each surface to one another. The rear surface of the rear surface may also have a moisture-wicking, fabric, padded layer that does not irritate the skin of a user when the device is worn by a user.

The top edge of the body is further comprised of at least one loop that receives at least one band that is preferably made from an elastic material, wherein the rear surface of the band is further comprised of the same padded material as the padded layer of the rear surface. In one embodiment, the band is one-size-fits-all and can stretch to fit a variety of users. However, in another embodiment, the band may be a two-piece band that has at least one fastener that allows a user to tighten or loosen the band around their waist and/or upper body. In another embodiment of the device, the bottom edge of the body is also comprised of at least one loop that receives at least one band of the same construction. In this manner, said band of the bottom edge functions as a leg-worn band that offers additional support to the body of the device.

To use the device, a user first places a drainage bag within the interior space of the body. Then, the user can secure both fasteners of the front surface and rear surface together to secure the bag within the body. Next, a user can then secure the at least one band around their waist and/or upper body. In an embodiment of the device with two bands the second band can also be secured around the thigh of the user. Once the bag is within the body of the device and the device is secured on the user, the user can then get dressed as normal. Because of the streamlined shape of the body, the bag is concealed from view and the user can also wear normal clothes due to this streamlined shape. In addition, the device prevents the bag from dragging or falling on the ground.

Accordingly, the body-worn drainage bag storage device of the present invention is particularly advantageous as it provides a device that allows a user to conceal a gall bladder drainage bag or other similar drainage bag of the like from view. Further, the device can be worn on the body of a user wherein the device conceals a gall bladder drainage bag or other similar drainage bag of the like from view. As a result, the device allows a user to wear normal clothes that still conceal the drainage bag. In this manner, the body-worn drainage bag storage device overcomes the limitations of existing body-worn drainage bags and methods of wearing said drainage bags known in the art.

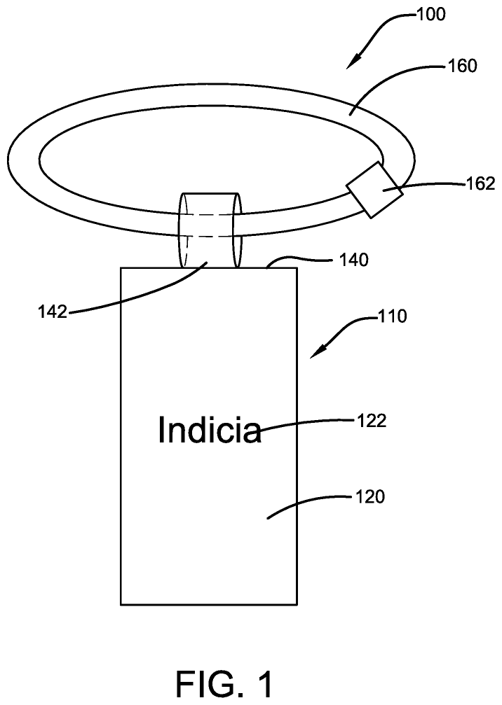

Referring initially to the drawings, FIG. 1 illustrates a perspective view of one potential embodiment of a body-worn drainage bag storage device 100 of the present invention in accordance with the disclosed architecture. The device 100 is primarily comprised of a body 110 that receives a drainage bag 20, wherein the body is further comprised of a front surface 120, a rear surface 130, and at least one loop 152 that receives at least one band 160. The body 110 is preferably manufactured from a durable fabric such as, but not limited to: vinyl, canvas, cashmere, chenille, chiffon, cotton, damask, jersey, lace, linen, wool, modal, polyester, satin, silk, spandex, suede, tweed, twill, velvet, acrylic, modacrylic, nylon, polypropylene, polyurethane, polyvinyl chloride, polyethylene, vinylidene, benzoate, aramid, rayon, acetate, triacetate, etc. The fabric material is further preferably waterproof or water-resistant. The body 110 may also be opaque, semi-transparent, or transparent. Additionally, the body 110 may be comprised of a plurality of indicia 122 such as, but not limited to: patterns, logos, emblems, images, symbols, designs, letters, words, characters, animals, advertisements, brands, etc. The body 110 can also be any color known in the art. The body 110 is preferably rectangular in shape, but may be any shape known in the art such that the body 110 can receive a drainage bag 20 within an interior space 170 of the body 110. In differing embodiments, the drainage bag 20 may be any body-attached drainage bag known in the art such as, but not limited to, a gall bladder drainage bag.

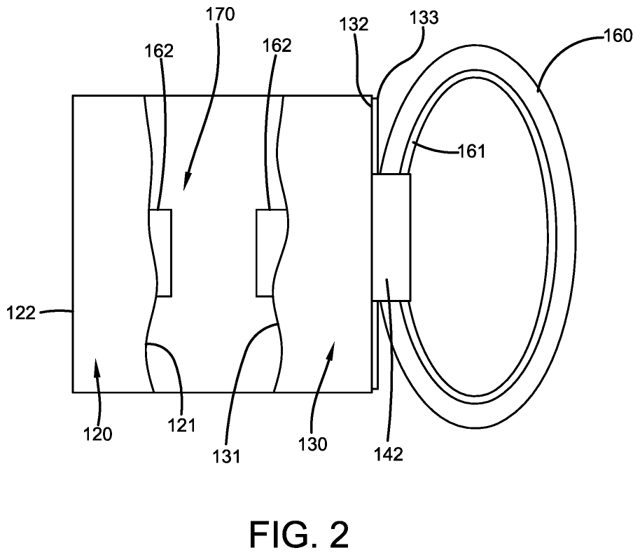

FIG. 2 illustrates a top view of one potential embodiment of a body-worn drainage bag storage device 100 of the present invention in accordance with the disclosed architecture. The rear surface 121 of the front surface 120 and the front surface 131 of the rear surface 130 are each further comprised of at least one fastener 162. In the preferred embodiment of the device 100, the fastener 162 of the rear surface 121 is a hook fastener and the fastener 162 of the front surface 120 is a loop fastener. Although, in differing embodiments, the fastener 162 may be any fastener known in the art such as, but not limited to: a magnetic fastener, a snap-button fastener, a zipper fastener, an adhesive fastener, etc. such that the front surface 120 can be secured to the rear surface 130. In this manner, a bag 20 can be secured within the interior space 170 of the body 110 while the device 100 is being used by securing the fasteners 162 of each surface 121, 131 to one another. The rear surface 132 of the rear surface 130 is further comprised of a padded layer 133. Said layer 133 is preferably manufactured from a moisture-wicking, soft fabric material that does not irritate the skin of a user 10 when the device 100 is worn by a user 10.

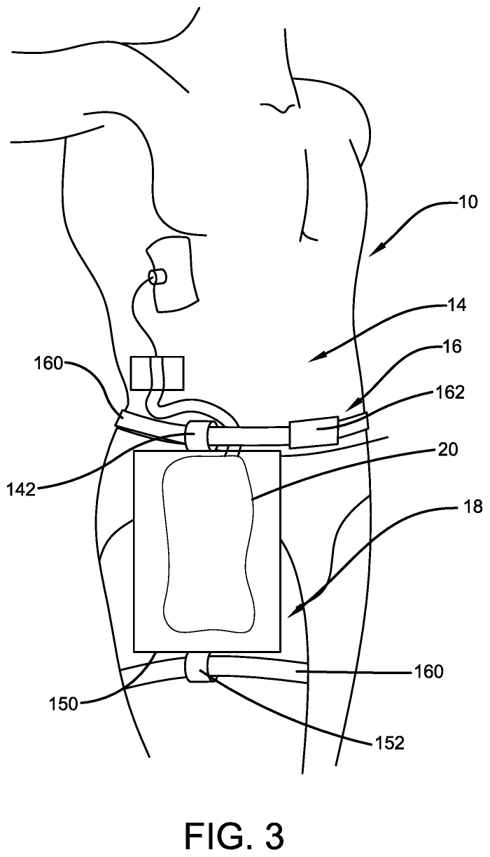

FIG. 3 illustrates a perspective view of one potential embodiment of a body-worn drainage bag storage device 100 of the present invention while being worn by a user 10 in accordance with the disclosed architecture. The top edge 140 of the body 110 is further comprised of at least one loop 142 that receives at least one band 160. The band 160 is preferably made from an elastic material, wherein the rear surface 161 of the band 160 is further comprised of the same padded material as the padded layer 133 of the rear surface 130. In one embodiment, the band 160 is one-size-fits-all and can stretch to fit a variety of users 10. However, in another embodiment, the band 160 may be a two-piece band 160 that has at least one fastener 162 such as, but not limited to: a buckle, a zipper, a slide, a belt loop, a magnet, etc. that allows a user 10 to tighten or loosen the band 160 around their waist 16 and/or upper body 14. In a differing embodiment, the bottom edge 150 of the body is also comprised of at least one loop 142 that receives at least one band 160. The band 160 is preferably made from an elastic material, wherein the rear surface 161 of the band 160 is further comprised of the same padded material as the padded layer 133 of the rear surface 130. In one embodiment, the band 160 is one-size-fits-all. However, in another embodiment, the band 160 be a two-piece band that has at least one fastener 162 such as, but not limited to: a buckle, a zipper, a slide, a belt loop, a magnet, etc. that allows a user 10 to tighten or loosen the band 160 around their thigh 18. In this manner, the leg-worn band 160 offers additional support to the body 110.

To use the device 100, a user 10 first places a drainage bag 20 within the interior space 170 of the body 110. Then, the user 10 can secure both fasteners 162 of the front surface 120 and rear surface 130 together to secure the bag 20 within the body 110. A user 10 can then secure the at least one band 160 around their waist 16 and/or upper body 14. In an embodiment of the device 100 with two bands 160, the second band 160 can also be secured around the thigh 18 of the user 10. Once the bag 20 is within the body 110 and the device 100 is secured on the user 10, the user 10 can then get dressed as normal. Because of the streamlined shape of the body 110, the bag 20 is concealed from view and the user 10 can also wear normal clothes due to this streamlined shape. In addition, the device 100 prevents the bag 20 from dragging or falling on the ground.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “body-worn drainage bag storage device” and “device” are interchangeable and refer to the body-worn drainage bag storage device 100 of the present invention.

Notwithstanding the forgoing, the body-worn drainage bag storage device 100 of the present invention and its various components can be of any suitable size and configuration as is known in the art without affecting the overall concept of the invention, provided that they accomplish the above-stated objectives. One of ordinary skill in the art will appreciate that the size, configuration and material of the body-worn drainage bag storage device 100 as shown in the FIGS. are for illustrative purposes only, and that many other sizes and shapes of the body-worn drainage bag storage device 100 are well within the scope of the present disclosure. Although the dimensions of the body-worn drainage bag storage device 100 are important design parameters for user convenience, the body-worn drainage bag storage device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A body-worn drainage bag storage device comprising:
a body having a front surface, a rear surface and a top edge further comprised of at least one loop;
at least one band;
at least one fastener; and
an interior space that is sized and configured to receive a body-worn drainage bag.

2. The body-worn drainage bag storage device of claim 1, wherein the at least one fastener is located on a rear surface of the front surface.

3. The body-worn drainage bag storage device of claim 1, wherein the at least one fastener is located on a front surface of the rear surface.

4. The body-worn drainage bag storage device of claim 1, wherein the at least one band is elastic.

5. The body-worn drainage bag storage device of claim 1, wherein the body is rectangular in shape.

6. A body-worn drainage bag storage device comprising:
at least one band having a padded layer;
a body comprised of a front surface having at least one fastener, a rear surface having at least one fastener, and a top edge having at least one loop that receives the at least one band; and
an interior space that is sized and configured to receive a body-worn drainage bag.

7. The body-worn drainage bag storage device of claim 6, wherein the at least one band is elastic.

8. The body-worn drainage bag storage device of claim 7, wherein the at least one band is further comprised of at least one fastener that allows the at least one band to be tightened or loosened.

9. The body-worn drainage bag storage device of claim 8, wherein the at least one fastener of the at least one band is a buckle, a zipper, a slide, a belt loop or a magnet.

10. The body-worn drainage bag storage device of claim 6, wherein the body is manufactured from a waterproof fabric.

11. The body-worn drainage bag storage device of claim 6, wherein the padded layer is manufactured from a moisture-wicking material.

12. The body-worn drainage bag storage device of claim 6, wherein the padded layer is manufactured from a material that does not irritate a skin of a user.

13. A body-worn drainage bag storage device comprising:
a first band having a padded layer;
a second band having a padded layer;
a body comprised of a front surface having a first fastener, a rear surface having a second fastener and layer of padding, a top edge having a first loop that receives the first band, and a bottom edge having a second loop that receives the second band; and
an interior space that is sized and configured to receive a body-worn drainage bag.

14. The body-worn drainage bag storage device of claim 13, wherein each of the padded layers of the first and second bands and the layer of padding of the body are manufactured from a moisture-wicking material.

15. The body-worn drainage bag storage device of claim 13, wherein each of the padded layers of the first and second bands and the layer of padding of the body are manufactured from a material that does not irritate a skin of a user.

16. The body-worn drainage bag storage device of claim 13, wherein each of the first and second bands is comprised of an elastic material.

17. The body-worn drainage bag storage device of claim 16, wherein each of the first and second bands is comprised of at least one fastener that allows each of the first and second bands to be tightened or loosened.

18. The body-worn drainage bag storage device of claim 17, wherein the at least one fastener is a select one of a buckle, a zipper, a slide, a belt loop or a magnet.

19. The body-worn drainage bag storage device of claim 13, wherein the body is manufactured from a waterproof fabric.

20. The body-worn drainage bag storage device of claim 13, wherein the first fastener is removably attached to the second fastener.
